(19) United States
(12) Patent Application Publication
(10) Pub. No.: US 2008/0108867 A1
(43) Pub. Date: May 8, 2008

(54) Devices and Methods for Ultrasonic Imaging and Ablation

(76) Inventor: Gan Zhou, Plano, TX (US)

Correspondence Address: HITT GAINES P.C., P.O. BOX 832570, RICHARDSON, TX, 75083, US

(21) Appl. No.: 11/739,301

(22) Filed: Apr. 24, 2007

Related U.S. Application Data
(63) Continuation-in-part of application No. 11/315,546, filed on Dec. 22, 2005.
(60) Provisional application No. 60/867,415, filed on Nov. 28, 2006.
(60) Provisional application No. 60/884,241, filed on Jan. 10, 2007.

(51) Int. Cl. A61B 1/00; A61B 18/18; A61N 1/00

(52) U.S. Cl. 600/104; 606/7; 606/41; 607/116

(57) ABSTRACT

Devices (i.e., catheters and guidewires) for, and methods of, ultrasonic imaging and ablation. In one embodiment, a device includes: (1) a fiber-optic bundle configured to carry laser light for ultrasonic imaging, each fiber of the fiber-optic bundle having a reflective layer oriented at an acute angle with respect thereto at a distal end thereof, (2) an elongated member associated with the fiber-optic bundle and configured to carry energy for ablation, the energy for ablation projecting past the distal end and (3) a photoacoustic layer associated with the each fiber of the fiber-optic bundle and configured to receive at least some of the laser light for the ultrasonic imaging and generate ultrasonic pressure waves in response thereto.

### CROSS-REFERENCE TO RELATED APPLICATION

The present application claims priority based on U.S. Provisional Application Ser. No. 60/867,415, entitled “Catheter for Ultrasonic Imaging and Laser Ablation,” filed on Nov. 28, 2006, by Zhou, and further based on U.S. Provisional Application Ser. No. 60/884,241, also entitled “Catheter for Ultrasonic Imaging and Laser Ablation,” filed on Jan. 10, 2007, by Zhou, commonly owned with the present application and incorporated herein by reference. The present application is also a continuation-in-part of U.S. patent application Ser. No. 11/315,546, entitled “Image-Guided Laser Catheter,” filed on Dec. 22, 2005, by Zhou, commonly owned with the present application and incorporated herein by reference.

### TECHNICAL FIELD OF THE INVENTION

The invention relates generally to the field of medical catheters and guidewires and more specifically to devices, taking the form of either catheters or guidewires, and methods for ultrasonic imaging and ablation.

### BACKGROUND OF THE INVENTION

In interventional cardiology, catheters and guidewires are often inserted into a patient's artery or vein to help accomplish tasks such as angioplasty or pacemaker or defibrillator lead insertion. For example, a balloon dilation catheter expands at a site of blood vessel occlusion and compresses the plaque and improves patency of the vessel. An intravascular ultrasound catheter provides a 360° view of the lateral cross section of a vessel. Different types of atherectomy procedures are performed using devices such as the rotablade, laser catheter, radio-frequency (RF) catheter or ultrasonic ablation catheter. The remarkably successful stents are deployed with the help of a balloon catheter.

Chronic total occlusion (CTO) is a disease that remains difficult to treat interventionally due to the inherent nature of the disease and the lack of adequate tools and devices. Some of the early devices, such as the Magnum™ guidewire (Schneider, Zurich, Switzerland), were made of a Teflon-coated steel shaft with an olive blunt tip. Results using this device in 800 chronic cases of CTO showed angiographic success in only 64% of the cases. One of the major failure modes was inability of the guidewire to advance.

The Kensey™ catheter (Theratech, Miami, Fla.) was a flexible polyurethane catheter with a rotating cam at the distal tip driven by an internal torsion guidewire at a speed of 10,000 rpm. Clinical evaluation in 11 patients with peripheral CTO diseases demonstrated only a 63% successful rate. The development of the device halted due to safety concerns.

The ROTACS™ low speed rotational atherectomy catheter (Oscor, Palm Harbor, Fla.) was made of several steel coils connected to a distal blunt tip of 1.9 mm. A motor drove the catheter rotation at 200 rpm. The catheter was unsuccessful due to safety concerns arising from the data that 30% of patients had extensive dissections.

The Excimer Laser Wire™ catheter (Spectranetics, Colorado Springs, Colo.) comprised a bundle of silica fibers that delivered excimer laser energy to the distal tip to ablate atherosclerotic plaque. In one clinical trial, the catheter was found to have a high rate of misalignment and perforation due to a stiff guidewire tip and a lack of guidance.

The Frontrunner™ catheter (LuMend, Redwood City, Calif.) is designed with a blunt tip designed to micro-dissect its way through a CTO. A bilaterally hinged distal tip assembly is manually opened and closed by the clinician to accomplish micro-dissection. The device has found some success in treating peripheral CTOs and also has a niche in treating coronary cases with refractory in-stent CTOs wherein the stent serves to confine and guide the device through the occlusion. However, the Frontrunner™ is not suitable for the majority of coronary CTO cases due to poor steerability and the lack of guidance.

The Safe Cross™ guidewire (Intraluminal Therapeutics, Carlsbad, Calif.) combines RF ablation capability with reflectometry at the distal tip. The optical reflectometry system provides a warning signal when the guidewire tip is too close to the vessel wall, and the RF ablation provides a way to cross hard calcified plaque. The device has had some success in recent clinical trials, but it is difficult to use and has yet to show widespread acceptance by interventionalists. The issue with the Safe Cross™ guidewire is that the optical reflectometry system generates a warning signal so frequently that leaves the operator at a loss as to what to do. Such a “negative” signal only tells the clinician what to avoid and fails to provide positive guidance for guidewire steering and advancement. Furthermore, there is no definitive indication of whether the guidewire tip is intra-luminal or extra-luminal. If for any reason the guidewire tip had accidentally perforated the vessel wall, the reflectometry signal would become useless.

Another way to provide a guidance signal for a catheter is to use laser-induced fluorescence. The healthy tissue of the artery wall and the atherosclerotic plaque attached to the wall have different fluorescent spectra or “signatures.” A system that detects this fluorescent signature should be able to tell whether the distal tip of the catheter is surrounded by healthy tissue or by plaque. A warning signal derived from laser induced fluorescence may have some advantages over the optical reflectometry signal, but the drawbacks are similar, namely, no geometric information about the diseased vessel.

A much more effective CTO intervention involves the use of imaging to guide the advancement of guidewires and catheters. Fluoroscopy is a well-established real-time external imaging modality. Fluoroscopy is used to guide many procedures, but its efficacy in CTO intervention has proven to be rather limited. Even with bi-plane projections, fluoroscopic images are hard to interpret for totally occluded vessel regions. Another issue with excessive dependence on fluoroscopy arises from the fact that CTO procedures are often time-consuming. Radiation safety as well as contrast fluid dosage are additional variables that the clinicians must monitor carefully during an already-stressful CTO intervention. Given these considerations, it is clear that an intravascular image-guided device would be highly valuable for CTO intervention.

A plurality of intravascular imaging devices have been developed to date. Angioscopy can supply visual information on the luminal surface, using a fiber bundle to illuminate the intraluminal space and also to collect reflected light to form an image. Angioscopy requires flushing the blood and replacing it with saline, a procedure that requires temporarily occluding the blood vessel and can cause prolonged ischemia to the heart. Because of this problem, angioscopy is used rarely other than for research purposes.

Intravascular ultrasound, or IVUS, can provide a cross-sectional image in a plane perpendicular to the catheter's axis and has become a very successful diagnostic tool in interventional cardiology and other medical applications. IVUS can image through blood with an acceptable range and has become a very successful diagnostic tool in interventional cardiology. In IVUS, an ultrasonic transducer is embedded in the distal end of an imaging catheter. The catheter is advanced through the vascular system to the target area. The transducer emits ultrasonic pulses and listens for echoes from the surrounding tissue to form a one-dimensional image. The catheter can be rotated to obtain two-dimensional imaging data or, alternatively, a solid-state IVUS with an annular array of transducers at the catheter distal surface can be used to perform 2D image scanning. Combined with a controlled pullback motion, the device can also obtain three-dimensional image data in a cylindrical volume centered on the catheter. While IVUS would at first appear to be an attractive solution for guiding the advancement of a guidewire through a CTO, existing IVUS catheters have proven difficult to advance through occluded regions of calcified tissue or tissue having a significant degree of fibrosis. For short occlusions, a clinician might be able to use a forward-looking IVUS to guide the advancement of the guidewire through the blockage, but even such forward-looking IVUS are still under development and not yet commercially available.

Optical coherence tomography is a relatively new imaging modality that has been considered for use in CTO intervention. The module uses low-coherence light interferometry to map out the optical absorption and scattering properties of the tissue under illumination. Optical coherence tomography provides image resolution that is about 10 times better than IVUS, but the imaging range is limited to a maximum of 3 to 4 millimeters. In addition, imaging through blood is very difficult even with carefully-chosen infrared wavelength for the light source. Without a significantly better imaging range, the microscopic resolution is of little usage to CTO guidance, as the most decisive clue that the clinicians can use during a procedure is the large-scale geometric feature that reveal the contour of the blood vessel wall.

U.S. Pat. No. 4,887,605 by Angelsen, et al., describes a laser catheter with an integrated ultrasound imaging module. A housing at the distal end of the catheter contains the ultrasonic transducer. An optical fiber is placed in a central through bore and delivers laser energy to the tissue to be treated. Unfortunately, this device would be difficult to advance through a CTO, because the area that contains the ultrasonic transducer apparently is bulky and lacks the ability to ablate plaque. In addition, Angelsen, et al., discloses no ability to perform forward imaging.

U.S. Pat. No. 4,587,972 by Morantte also described a combined ablation and ultrasound-imaging catheter. The catheter contains a fiber bundle for laser delivery and ultrasound transducers that emits in the forward direction. However, Morantte's catheter is apparently bulky and difficult to advance through CTOs.

Accordingly, what is needed in the art is a device, taking the form of either a catheter or a guidewire, for ultrasonic imaging and ablation that overcomes at least some of the disadvantages of the devices described above. What is further needed in the art is a method of operating such a device that is particularly suited to treating CTO.

### SUMMARY OF THE INVENTION

To address the above-discussed deficiencies of the prior art, the invention provides, in one aspect, devices (i.e., catheters and guidewires) for ultrasonic imaging and ablation. In one embodiment, a device includes: (1) a fiber-optic bundle configured to carry laser light for ultrasonic imaging, each fiber of the fiber-optic bundle having a reflective layer oriented at an acute angle with respect thereto at a distal end thereof, (2) an elongated member associated with the fiber-optic bundle and configured to carry energy for ablation, the energy for ablation projecting past the distal end and (3) a photoacoustic layer associated with the each fiber of the fiber-optic bundle and configured to receive at least some of the laser light for the ultrasonic imaging and generate ultrasonic pressure waves in response thereto.

In another embodiment, a device includes: (1) a fiber-optic bundle configured to carry laser light for ultrasonic imaging, (2) an elongated member associated with the fiber-optic bundle and configured to carry energy for ablation, (3) a distal cap having a glass element aligned with the fiber-optic bundle to receive the laser light for the ultrasonic imaging, (4) a reflective layer oriented at an acute angle with respect to the glass element and configured substantially to reflect the laser light for the ultrasonic imaging, the energy for ablation projecting past the reflective layer and (5) a photoacoustic layer associated with the glass element and configured to receive at least some of the laser light for the ultrasonic imaging and generate ultrasonic pressure waves in response thereto.

In another aspect, the invention is directed to methods of ultrasonic imaging and ablation. In one embodiment, a method includes: (1) causing laser light for ultrasonic imaging to be carried through a fiber-optic bundle of a device, each fiber of the fiber-optic bundle having a reflective layer oriented at an acute angle with respect thereto at a distal end thereof, (2) causing energy for ablation to be carried through an elongated member associated with the fiber-optic bundle, the energy for ablation projecting past the distal end and (3) causing a photoacoustic layer associated with the each fiber of the fiber-optic bundle to receive at least some of the laser light for the ultrasonic imaging and generate ultrasonic pressure waves in response thereto.

In another embodiment, a method includes: (1) causing laser light for ultrasonic imaging to be carried through a fiber-optic bundle of a device, (2) causing energy for ablation to be carried through an elongated member associated with the fiber-optic bundle, (3) causing the laser light for the ultrasonic imaging to be received into a distal cap having a glass element aligned with the fiber-optic bundle, (4) causing the laser light for the ultrasonic imaging to be substantially reflected off a reflective layer oriented at an acute angle with respect to the glass element, (5) causing the energy for ablation to be projected past the reflective layer and (6) causing at least some of the laser light for the ultrasonic imaging to be received by a photoacoustic layer associated with the glass element and converted into ultrasonic pressure waves.

The foregoing has outlined certain features of the invention so that those skilled in the pertinent art may better understand the detailed description of the invention that follows. Additional features of the invention will be described hereinafter that form the subject of the claims of the invention. Those skilled in the pertinent art should appreciate that they can readily use the disclosed conception and specific embodiment as a basis for designing or modifying other structures for carrying out the same purposes of the invention. Those skilled in the pertinent art should also realize that such equivalent constructions do not depart from the scope of the invention.

### BRIEF DESCRIPTION OF THE DRAWINGS

For a more complete understanding of the invention, reference is now made to the following descriptions taken in conjunction with the accompanying drawings, in which:

FIG. 1 is a schematic view of one embodiment of a catheter constructed according to the principles of the invention;

FIGS. 2A and 2B together show one example of an arrangement of optical fibers inside a catheter;

FIG. 3 shows a cross-sectional view of one embodiment of the catheter constructed according to the principles of the invention;

FIG. 4 is a cross-sectional view of one embodiment of a distal cap of the catheter of FIG. 3;

FIG. 5 is an isometric view of one embodiment of a distal segment of the catheter of FIG. 3;

FIG. 6 shows a cross-sectional view of another embodiment of the catheter constructed according to the principles of the invention;

FIG. 7 is a cross-sectional view of one embodiment of a distal cap of the catheter of FIG. 6;

FIG. 8 is an isometric view of a distal segment of the catheter of FIG. 6;

FIG. 9 shows one embodiment of a distal segment of the catheter of FIG. 3 that incorporates one or more ultrasonic sensors;

FIG. 10 is a schematic view of another embodiment of a catheter constructed according to the principles of the invention;

FIG. 11 illustrates one embodiment of a connector at a proximal end of the catheter of FIG. 10;

FIG. 12 illustrates one embodiment of a distal tip of the catheter of FIG. 10;

FIGS. 13A, 13B and 13C illustrate isometric views of alternative embodiments of a distal tip of a photoacoustic fiber;

FIG. 14 details a part of a catheter's distal tip;

FIG. 15 illustrates a cross-sectional view of one embodiment of a distal tip of the catheter of FIG. 10;

FIG. 16 illustrates a sectional view of the distal tip of FIG. 15; and

FIG. 17 details a part of a catheter's distal tip.

### DETAILED DESCRIPTION

Several embodiments of the invention will now be described. Various structures, arrangements, relationships and functions may be asserted as being associated with or necessary to certain of the several embodiments. Those skilled in the pertinent art should understand, however, that those structures, arrangements, relationships and functions need not be associated with or necessary to the invention in general.

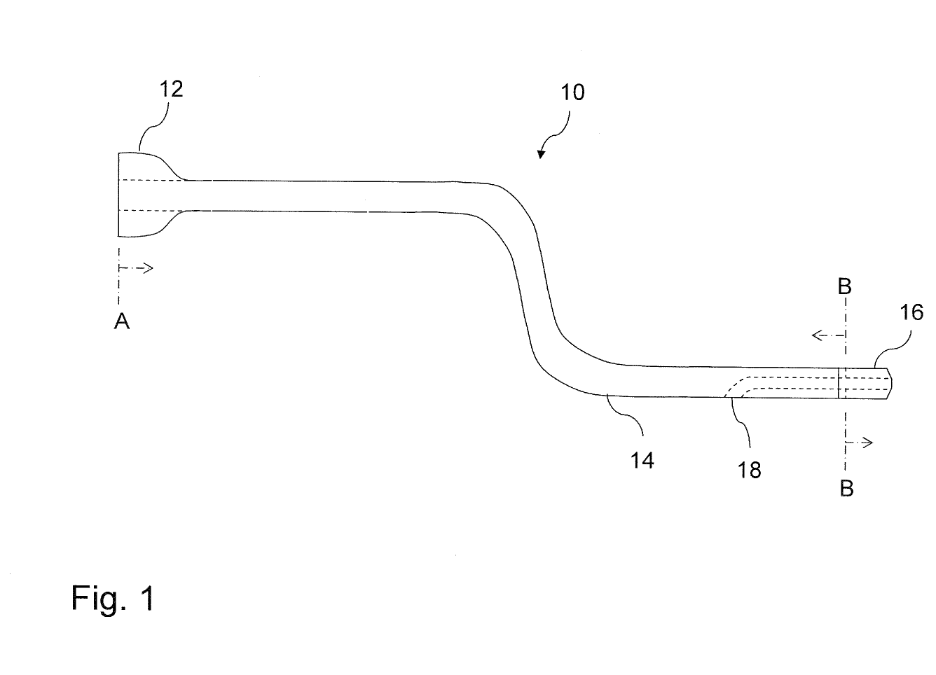

Referring initially to FIG. 1, illustrated is a schematic view illustrating one embodiment of a catheter 10 constructed according to the principles of the invention. The catheter 10 includes an elongated main body 14, a connector 12 at a proximal end thereof and a cap 16 affixed to the main body 14 at a distal end thereof. A guidewire lumen 18 is associated with the catheter 10. A plurality of optical fibers is embedded inside the catheter main body 14. The optical fibers extend from section A to section B in FIG. 1.

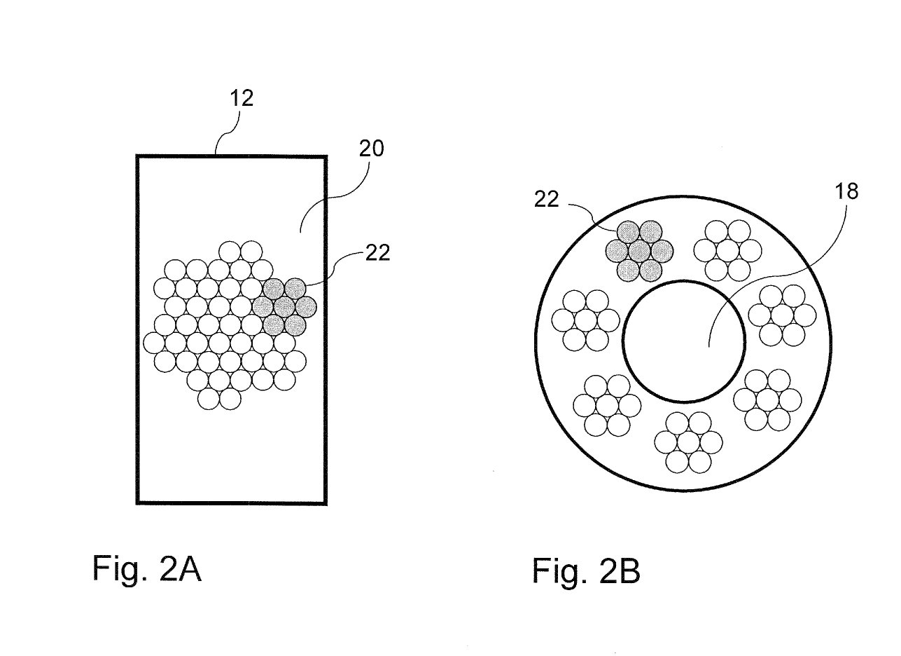

FIGS. 2A and 2B together show one embodiment of a way optical fibers may be arranged inside the catheter. FIG. 2A shows the cross-sectional view of the catheter at section A of FIG. 1, looking into the catheter body. The optical fibers are bundled together to fill an approximately circular area, forming a fiber bundle 20. The fiber bundle 20 is divided into a plurality of subgroups, including a subgroup 22. The optical fibers in the subgroup 22 are shaded in FIGS. 2A and 2B for the purpose of visual clarity. FIG. 2B shows the cross-sectional view of the catheter at section B of FIG. 1, looking into the catheter main body 14. The fiber subgroups are approximately evenly spaced inside the catheter and centered around the guidewire lumen 18. At the proximal end of the catheter, two types of laser energy are coupled into the fiber bundle 20. A first laser, such as a 308 nanometer laser, provides laser energy into the entire bundle and directed to the catheter distal tip for tissue ablation. A second laser, such as a 523 nanometer Q-switched pulse laser, provides laser energy into a fiber subgroup and directed to the catheter's distal tip for ultrasonic wave excitation. The second laser scans the fiber subgroups, illuminating them in sequence, so that all the fiber subgroups are illuminated over a period of time.

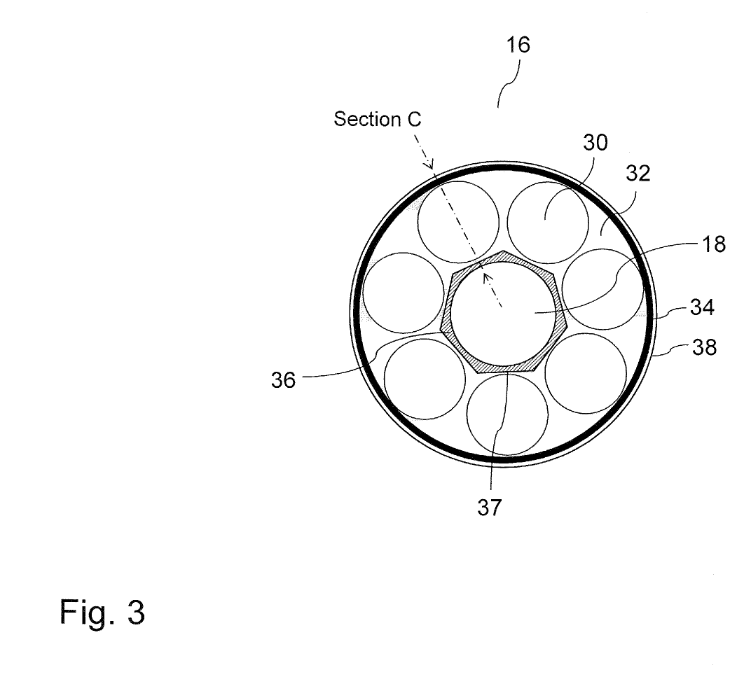

FIG. 3 shows one embodiment of the cross-sectional view of the catheter at section B of FIG. 1, looking into the distal cap 16. A plurality of polished glass rods 30 (a glass rod being one type of glass element) are placed around the center guidewire lumen 18. The plurality of glass rods are the same as the plurality of fiber subgroups 22 in the catheter main body 14. The center of each glass rod is aligned with the center of a corresponding fiber subgroup. The size of a glass rod 30 is slightly larger than the size of a fiber subgroup 22, so that any light emerging from the fiber subgroup 22 is substantially captured by the glass rod 30. One way to fabricate the glass rod 30 is by using optical fibers having the appropriate core and cladding diameters and cutting and polishing the ends to the appropriate length and surface finish. The glass rod 30 may be a short segment of optical fiber that can guide and confine light propagation, like a waveguide. In the illustrated embodiment, the glass rod 30 is embedded in index-matching epoxy 32, whose refractive index is substantially the same as that of the cladding layer of the glass rod 30. At the center of the distal cap 16 is a mandrel 36 with a hollow core that forms the distal part of the guidewire lumen 18. The mandrel's polygonal outer surfaces 37 are deposited with high reflectivity coatings for the second laser (the ultrasound excitation laser). The glass rods are enclosed by a photoacoustic layer 34 and a protective layer 38. The photoacoustic layer 34 can be made from a material that is highly absorptive to the second laser and has a large thermal expansion coefficient. The material may be an elastomer, such as polydimethylsiloxane (PDMS) mixed with the appropriate amount of carbon black powder. Other types of photoacoustic material suitable for use in the photoacoustic layer 34 are familiar to those skilled in the pertinent art. For example, the photoacoustic layer 34 may be a thin layer of metal film, doped glass, doped plastic, gel-like material or even a liquid layer such as blood.

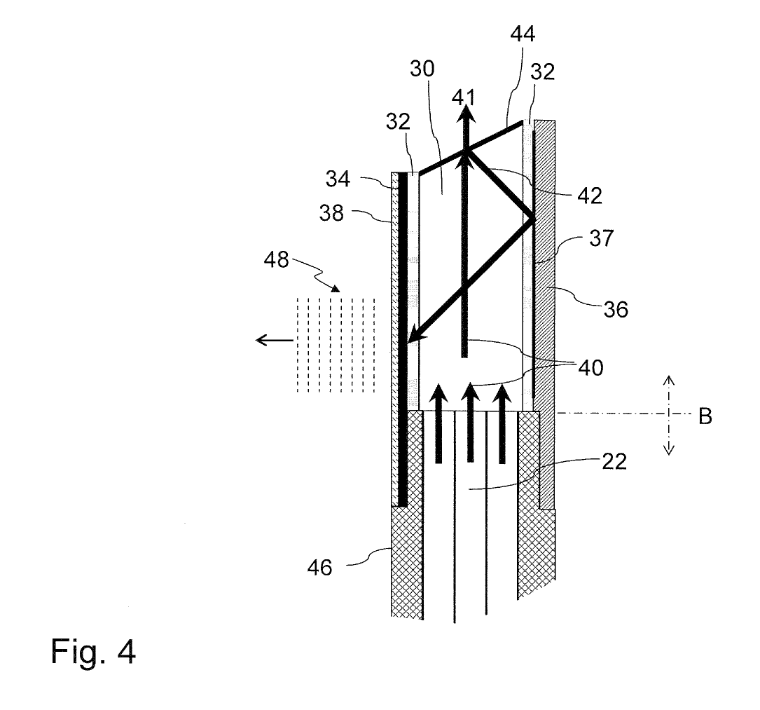

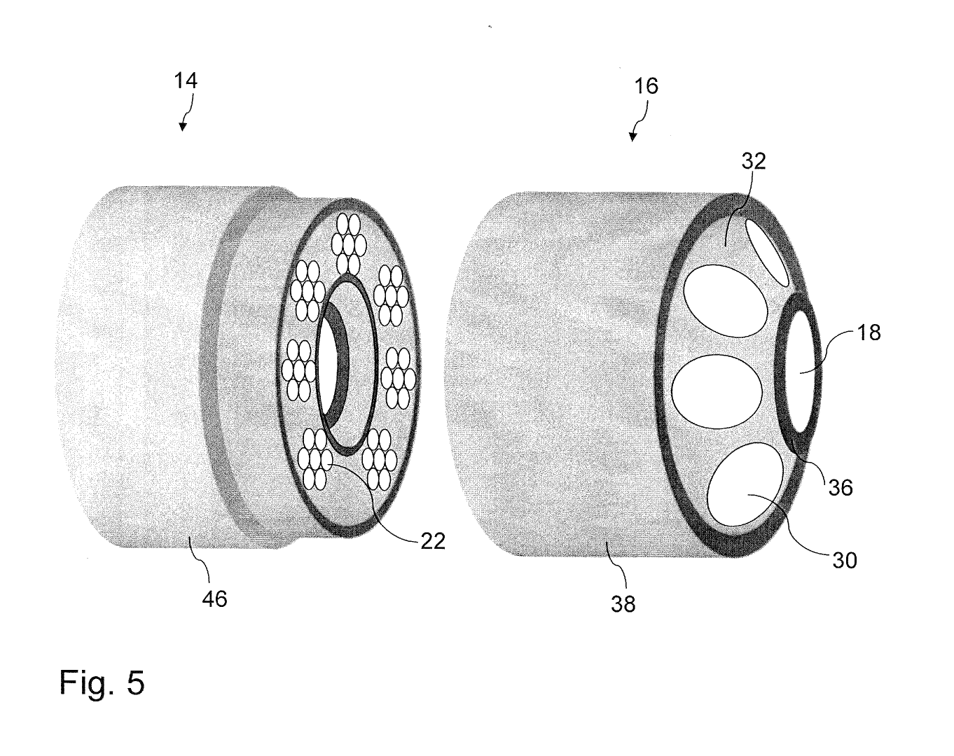

FIG. 4 is a cross-sectional view of the distal cap 16 at section C indicated in FIG. 3. As is illustrated in FIG. 4, the glass rod 30 is polished at a non-perpendicular wedge angle at the tip of the catheter, whereas it is polished at the perpendicular angle (or zero-wedge angle) on the opposite side. The fiber subgroup 22 in the main body of the catheter is butt-coupled to glass rod 30, with or without any index-matching layer in between. The two coupling surfaces can also be deposited with anti-reflection coatings to minimize reflection loss. Laser light 40 emerging from the fiber subgroup 22 is captured by glass rod 30, and it propagates toward the catheter tip. A dichroic optical coating layer 44 is deposited at the wedged surface of glass rod 30, such that light 41 at the wavelength of the first laser (the ablative laser) substantially transmits through the coating layer 44, whereas laser light 42 at the wavelength of the second laser (the ultrasound excitation laser) substantially reflects off the coating layer 44. As pointed out above, the transmitted laser 41 can be, for instance, a 308 nanometer laser that ablates the tissue near the catheter tip, whereas the reflected laser 42 can be, for example, a 523 nanometer pulsed laser that is used for the purpose of photoacoustic generation. The photoacoustic laser light 42 is subsequently reflected by the coating 37 on the flat surface of mandrel 36 before it reaches the photoacoustic layer 34. Note that the wedge angle of glass rod 30 should be large enough, for instance from 10° to 40°, such that the reflected laser 42 is no longer inside the waveguide acceptance angle of the glass rod 30. Because the refractive index differences among the index-matching epoxy 32, the core and cladding of the glass rod 30 are very small, laser beam 42 experiences only a small loss as it travels through the glass rod 30 and the index-matching epoxy to reach the photoacoustic layer 34. The photoacoustic layer 34 includes a material that is highly absorptive at the wavelength of laser 42. The absorbed laser energy causes a rapid thermal-elastic expansion of the layer 34 and generates an ultrasonic pressure wave 48 that penetrates into the surrounding fluid and/or tissue. FIG. 5 is an isometric view of the distal segment of the catheter 10. The distal cap 16 is shown to be separated from the catheter main body 14 for illustration purpose. The distal cap 16 may be affixed securely to the main body 14 by a mechanical fitting using appropriate adhesives. As has been discussed, the distal cap 16 contains an array of glass rods 30 with associated surfaces and coatings. However, those skilled in the pertinent art are aware of other ways to implement the distal cap 16. For example, each glass rod in the array of glass rods 30 in FIG. 5 does not have to be cylindrical in shape; it may be a rectilinear plate instead. In another example, the array of glass rods 30 in FIG. 5 can be replaced with a ring-shaped glass collar (not shown) having a conical surface at the distal end and a flat surface at the opposite end. In this embodiment, an appropriate reflective coating is located on the conical surface, and a photoacoustic layer is located on the outside wall of the glass collar.

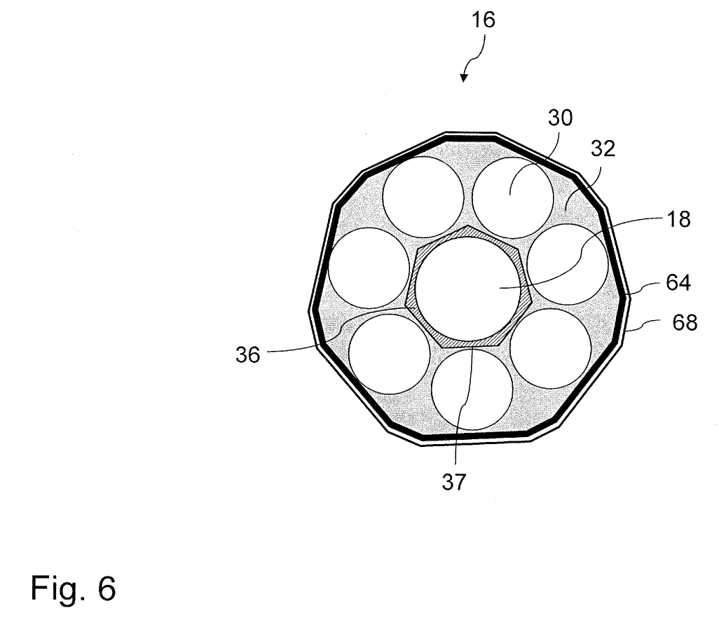

FIG. 6 shows another example embodiment of the distal cap 16. Compared to the design shown in FIG. 3, the main difference of the distal cap shown here is that the photoacoustic layer 64 and the protective layer 68 are polygonal, as opposed to circular. The polygonal provides a flat emission surface, as opposed to a convex emission surface, for the laser-generated ultrasound. The propagation of photoacoustic emission from a flat surface can be advantageously less divergent compared to that from a convex surface. Based on the same principal, another alternative is to make the photoacoustic emission area slightly concave so that the photoacoustic wave propagates into surrounding fluid and/or tissue with minimal divergence.

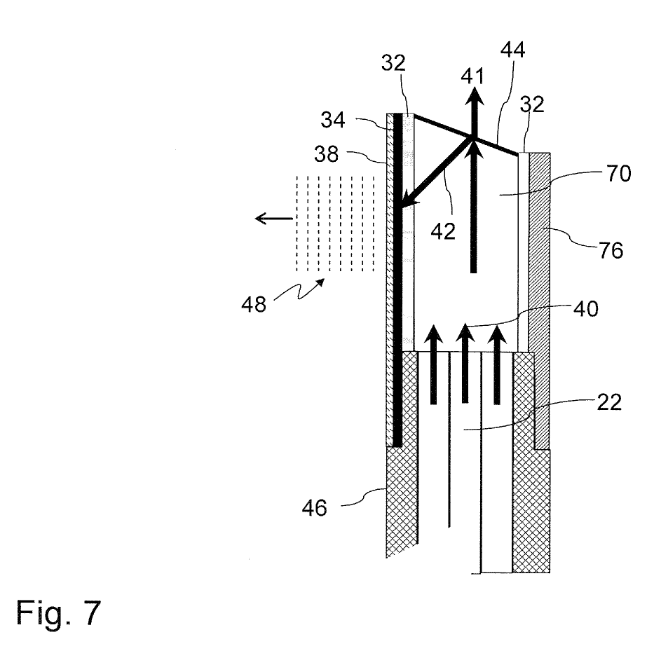

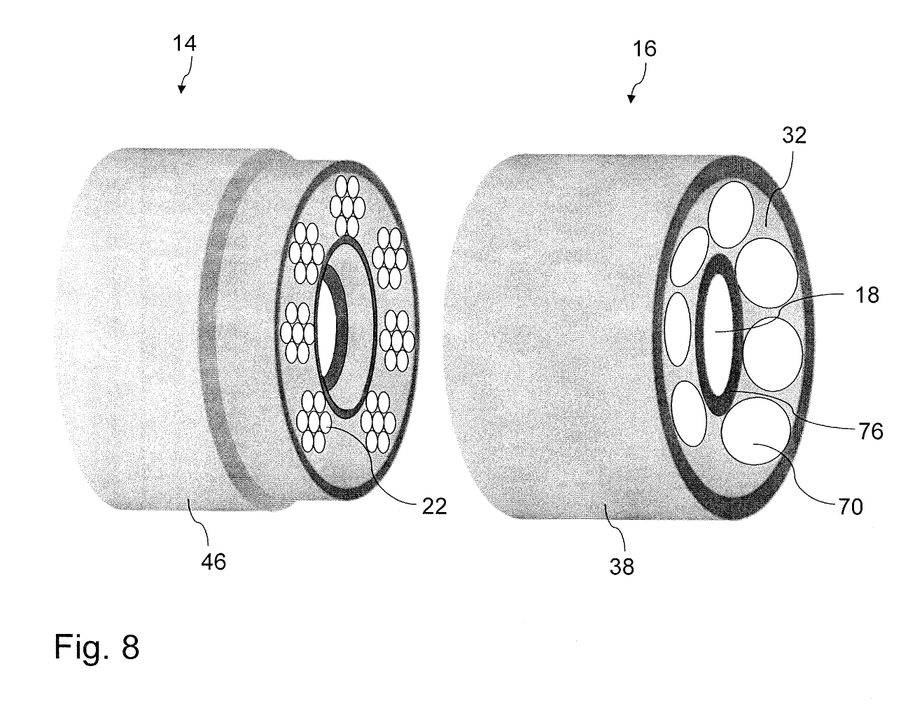

FIG. 7 shows yet another example embodiment of the distal cap 16. Similar to FIG. 4, FIG. 7 is a cross-sectional view of the distal cap 16 at section C indicated in FIG. 3. As is illustrated here, the glass rod 70 is polished at a non-perpendicular wedge angle at the tip of the catheter, whereas the glass rod 70 is polished at the perpendicular angle on the opposite side. The fiber subgroup 22 in the main body of the catheter is butt-coupled to glass rod 70, with or without any index-matching layer in between. The two coupling surfaces can also be deposited with anti-reflection coatings to minimize reflection loss. Light 40 emerging from the fiber subgroup 22 is captured by glass rod 70 and it propagates toward the catheter tip. A dichroic optical coating layer 44 is deposited at the wedged surface of glass rod 70, such that light 41 at the ablative wavelength substantially transmits through the coating layer 44, whereas light 42 at the photoacoustic wavelength substantially reflects off the coating layer 44. The laser pulse 42 reaches the photoacoustic layer 34 and is substantially absorbed there. Note that the wedge angle of glass rod 70 should be large enough, for example from 10° to 40°, such that the reflected laser 42 is no longer inside the waveguide acceptance angle of glass rod 70. Because the refractive index differences among the index-matching epoxy 32, the core and cladding of the glass rod 70 are very small, laser beam 42 experiences only a small loss as it travels through the glass rod and the index-matching epoxy to reach the photoacoustic layer 34. The photoacoustic layer 34 includes a material that is highly absorptive at the wavelength of laser 42. The absorbed laser energy causes a rapid thermal-elastic expansion of the layer 34 and generates an ultrasonic pressure wave 48 that penetrates into the surrounding fluid and/or tissue. Note that the mandrel 76 here does not need to have a polygonal outer surface having a high-reflectivity coating, because laser beam 42 does not reach it in this embodiment. FIG. 8 is an isometric view of the distal segment of the catheter corresponding to the embodiment of FIG. 7. Again, the distal cap 16 is shown as being separated from the catheter main body 14 for illustration purpose. The distal cap 16 can be affixed securely to the main body 14 by a mechanical fitting (not shown) using appropriate adhesives. The distal tip of the catheter in this embodiment has a concave shape. As has been discussed, the distal cap contains an array of glass rods 70 with associated surfaces and coatings. However, those skilled in the pertinent art are aware of other ways to implement the distal cap. For example, each of the glass rod 70 in FIG. 8 does not have to be cylindrical in shape; it may be a rectilinear plate instead. In another example, the array of glass rods 70 in FIG. 8 can be replaced with a ring-shaped glass collar (not shown) having a conical surface at the distal end and a flat surface at the opposite end. In this embodiment, an appropriate reflective coating is located on the conical surface, and a photoacoustic layer is located on the outside wall of the glass collar.

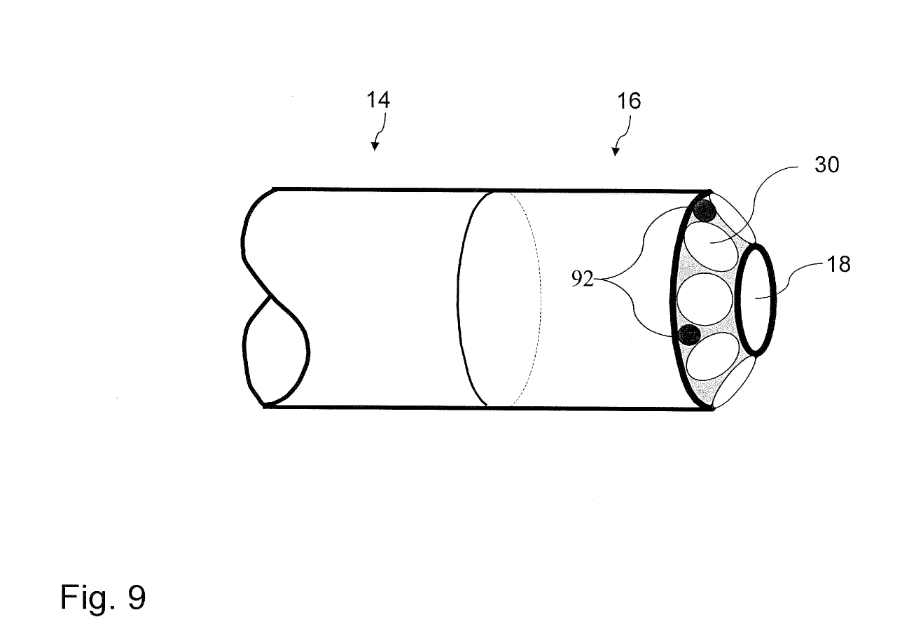

FIG. 9 shows one embodiment of a distal segment of the catheter 10 that incorporates one or more ultrasonic sensors 92. Ultrasonic echo from the nearby tissue can be received by the ultrasonic sensors 92 and used for imaging purposes. It is well understood in relevant art that the time-domain ultrasonic echo signal from the surrounding tissue can be processed and combined to form an ultrasonic image. These ultrasonic sensors 92 can be implemented in a variety of different ways, including using piezoelectric materials, PVDF membranes or fiber optical interferometers.

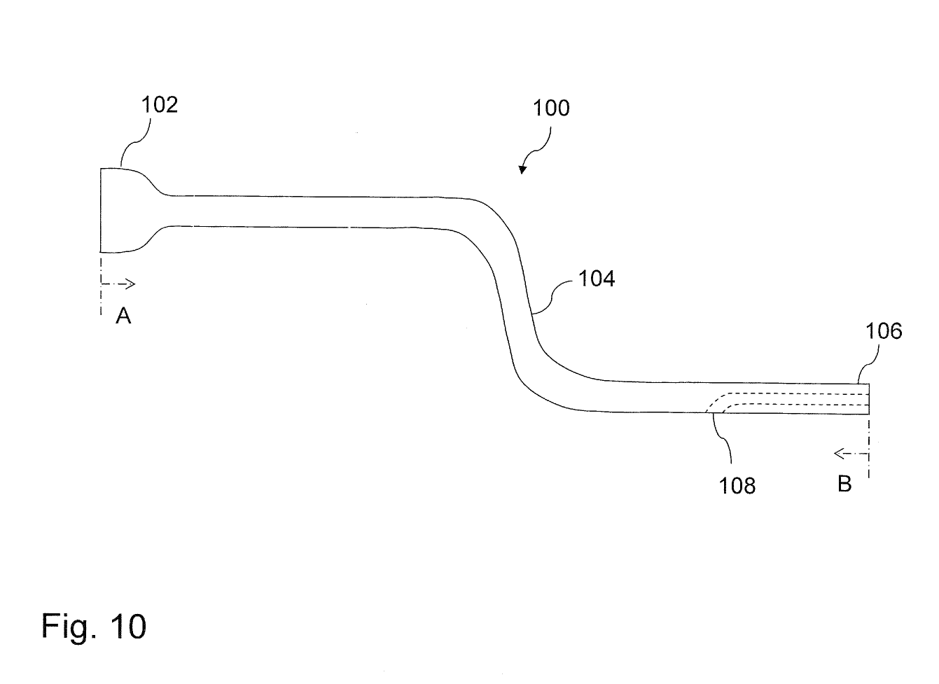

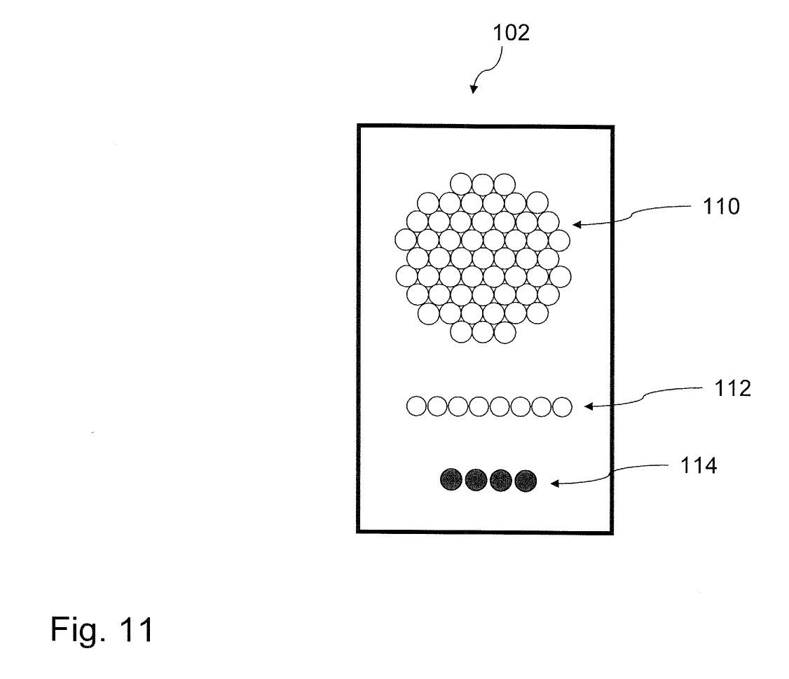

FIG. 10 is a schematic view illustrating another embodiment of the catheter of the invention. The catheter 100 includes an elongated body 104, a connector 102 at the proximal end and a distal segment 106. A guidewire lumen 108 is present in the catheter. A plurality of optical fibers and other components are embedded inside the catheter 100. FIG. 11 illustrates one embodiment of the connector 102 by showing the cross-sectional view of the catheter 100 at section A of FIG. 10. The optical fiber bundle 110 (called ablative fibers) form an approximately circular area. Ablative laser energy, such as that from a 308 nanometer laser, is coupled into the bundle 110 and directed to the catheter's distal tip for tissue ablation. The optical fiber array 112 (called photoacoustic fibers) receive and carry a second laser, such as a 523 nanometer Q-switched pulse laser, for the purpose of ultrasound excitation at the distal tip of catheter 100. The ultrasound excitation laser can be scanned to enter the fiber in the array 112 in a sequential manner, so that the entire array is accessed over a predetermined period of time. A group of ultrasonic sensors 114 are also present in the catheter 100, they are shaded in the drawing for the purpose of visual clarity. Note that the sensors 114 do not necessarily have a round shape like shown in FIG. 11. The actual sensor interface shown in FIG. 11 can be either electrical or optical, depending on the particular implementation. As pointed out earlier, these ultrasonic sensors can be implemented in a variety of different ways, including using piezoelectric ceramics, polyvinylidene fluoride (PVDF) membranes or by fiber optical interferometers. A fiber optic ultrasonic sensor has the advantage of being relatively small, e.g., 25 microns to 250 microns in diameter. Such small sensors can help keep the overall catheter miniature and flexible. Generally speaking, the connector 102 is plugged into a console that communicates with the catheter 100 optically and electrically.

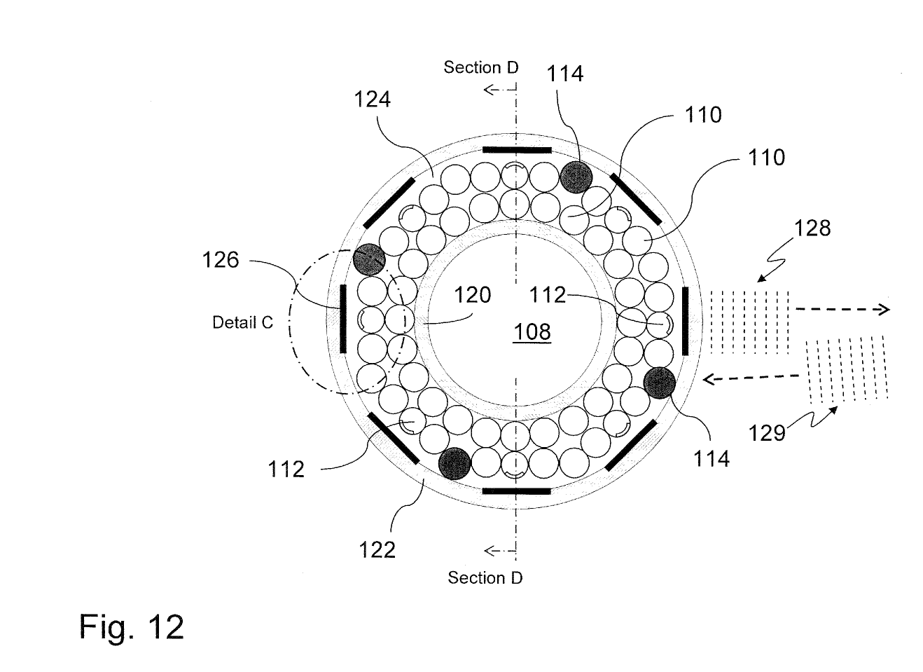

FIG. 12 illustrates one embodiment of the distal tip of the catheter 100 by showing the view at cross-section B in FIG. 10. The ablative fibers 110 are now dispersed inside the catheter around the guidewire lumen 108. The photoacoustic fibers 112 are also dispersed and positioned around the guidewire lumen 108 so that they have approximately equal angular separation from each other. The tip of the photoacoustic fibers 112 are modified somewhat as will be described later. The sensors 114 are positioned inside the catheter so that there is always one sensor in proximity to a photoacoustic fiber, although two or more photoacoustic fibers could share the same sensor. The catheter has an inner wall 120, an outer wall 122 and encapsulant 124 which fills the space between the optical fibers and the inner and outer walls. In one embodiment, the encapsulant can be an optical adhesive with a refractive index equal to or higher than that of the core of optical fibers. Near the outer wall 122 is a photoacoustic layer 126. The photoacoustic layer 126 can be made from a material that is highly absorptive to the ultrasound excitation laser and has a large thermal expansion coefficient. One example of such material is an elastomer such as PDMS mixed with an appropriate amount of carbon black powder. Laser pulses emerging from the side a photoacoustic fiber 112 is significantly absorbed by the photoacoustic layer 126. The absorbed laser energy causes a rapid thermoelastic expansion of the photoacoustic layer 126 and generates an ultrasonic pressure wave 128 that penetrates into the surrounding fluid and/or tissue. An ultrasonic echo 129 from the surrounding tissue is detected by a sensor 114 which is in proximity to that photoacoustic fiber 112.

As has been described, the catheter of the invention may employ laser light to ablate occlusions or tissue. However, those skilled in the pertinent art are aware of other ablation techniques or mechanisms. For example, it is known that RF energy may be used to ablate occlusions or tissue. It is also known that ultrasonic energy from a piezoelectric device can also be used to ablate tissue. It is further known that occlusions or tissue may be ablated mechanically, perhaps by a very small drill. Therefore, the scope of the invention includes catheters that can ablate by RF, ultrasonic or mechanical energy. In the case of RF ablation, some or all of the ablative fibers 110 are replaced by RF waveguides, typically taking the form of conductive wires. In the case of ultrasonic ablation, some or all of the ablative fibers 110 are replaced by wires carrying electrical pulses and terminating in one or more piezoelectric elements. In the case of mechanical ablation, some or all of the ablative fibers 110 are replaced by flexible drive shafts. Such drive shafts project from the distal end of the catheter and terminate in an ablating member, such as an auger, spade or grinding bit. Those skilled in the pertinent art are familiar with the wide variety of structures that may be employed to perform mechanical ablation. The invention encompasses all such and later-developed structures. Finally, those skilled in the pertinent art will recognize that a catheter constructed according to the principles of the invention may employ multiple ablative techniques or mechanisms, namely a combination of laser light, RF, ultrasonic and/or mechanical.

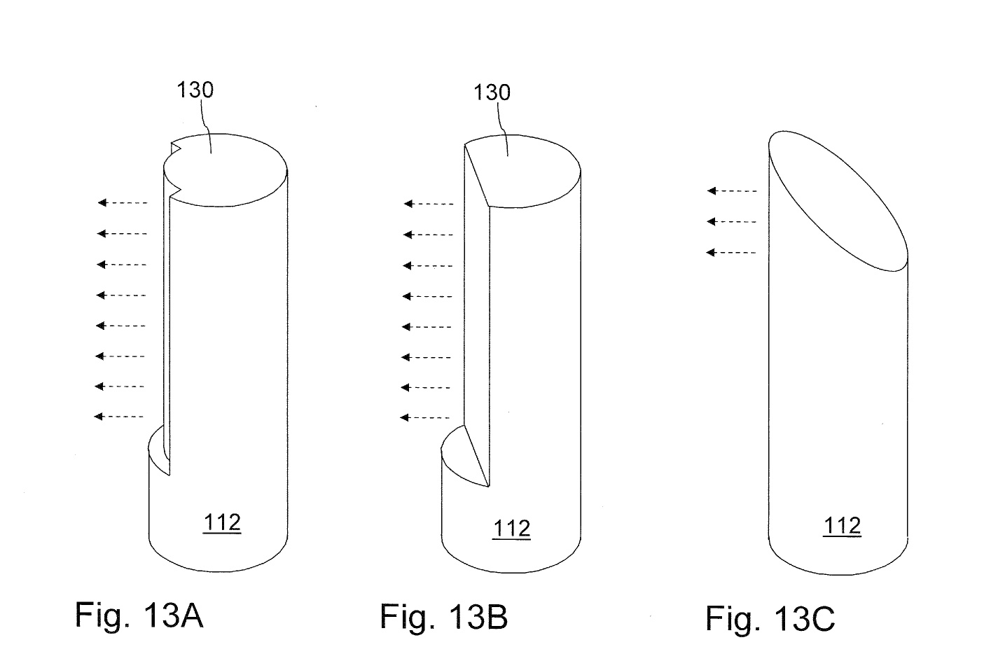

FIGS. 13A, 13B and 13C illustrate several different example embodiments by which the distal tip of a photoacoustic fiber 112 can be modified to allow laser light to exit from the side. FIG. 13A shows one embodiment where a section of the fiber 112 cladding is removed to expose the core. When the fiber 112 is immersed in a material with index of refraction equal to or higher than the core, light propagating inside the fiber 112 begins to exit to the side that has no cladding. The endface 130 of the fiber 112 can be optionally coated with a high reflectivity coating to further enhance the percentage of light exiting to the side. FIG. 13B shows an embodiment where a section of the circumference of the fiber 112 is polished flat to expose the core. Again, when the fiber 112 is immersed in a material with index of refraction equal to or higher than the core, light propagating inside the fiber 112 begins to exit to the side that has no cladding. The endface 130 of the fiber 112 can also be optionally coated with a high reflectivity coating to enhance the percentage of light exiting to the side. FIG. 13C shows the embodiment where the photoacoustic fiber tip is polished at a wedge angle (such as 50°). When the tip of this fiber is in contact with a low refractive index material (such as air), light propagating in the fiber is significantly reflected by the wedged endface and exit to the side direction as shown.

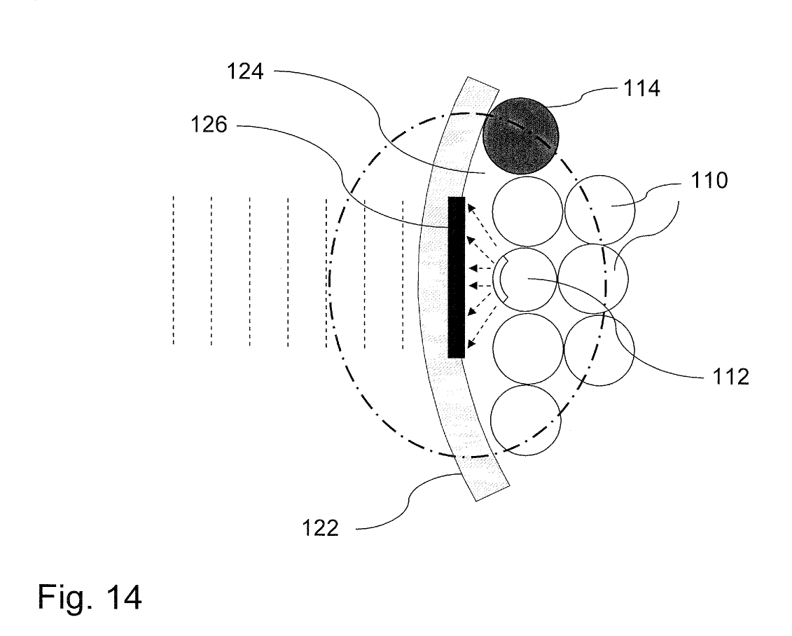

FIG. 14 further illustrates the detail C of the catheter distal tip shown in FIG. 12. Laser light exiting from the side of photoacoustic fiber 112 is absorbed by the photoacoustic layer 126 and generates an ultrasonic wave that penetrates into the surrounding medium.

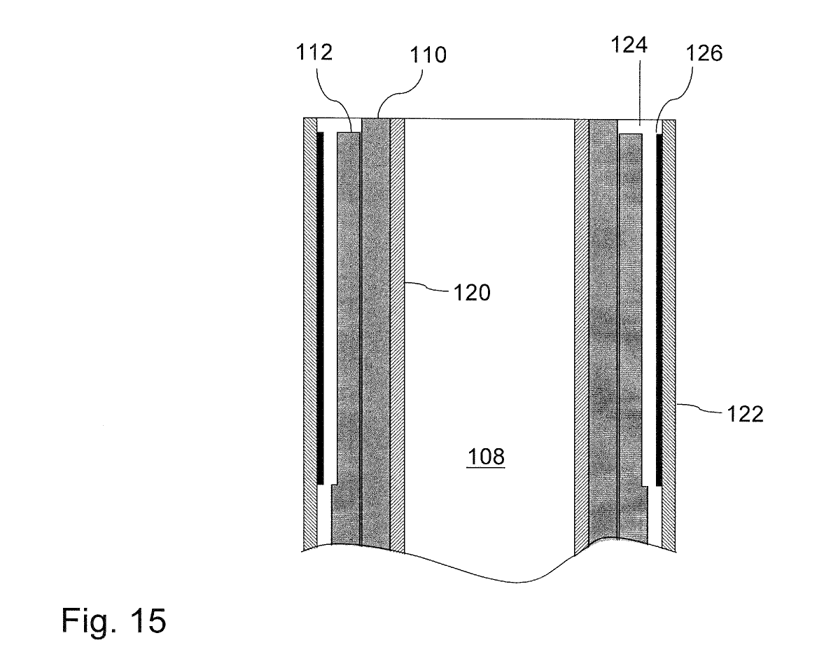

FIG. 15 further illustrates the view at cross-section D of the catheter distal tip shown in FIG. 12. Note that in the particular embodiment shown in FIG. 15 the tips of photoacoustic fibers 112 are recessed slightly from that of the ablative fibers 110. This may in some cases protect against abrasion to the optical coating that may be present on the end face of the photoacoustic fibers.

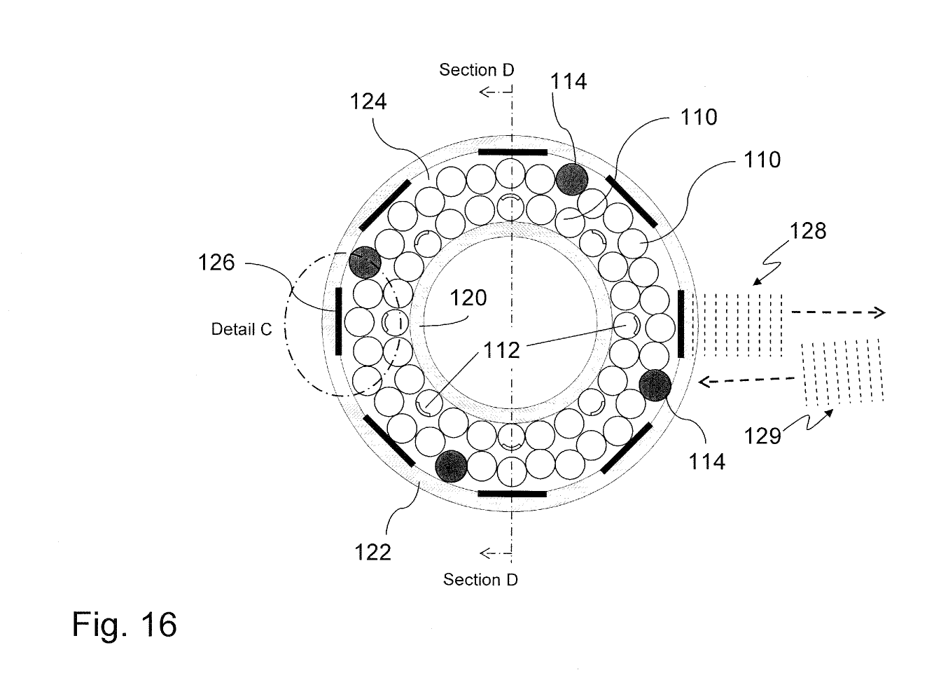

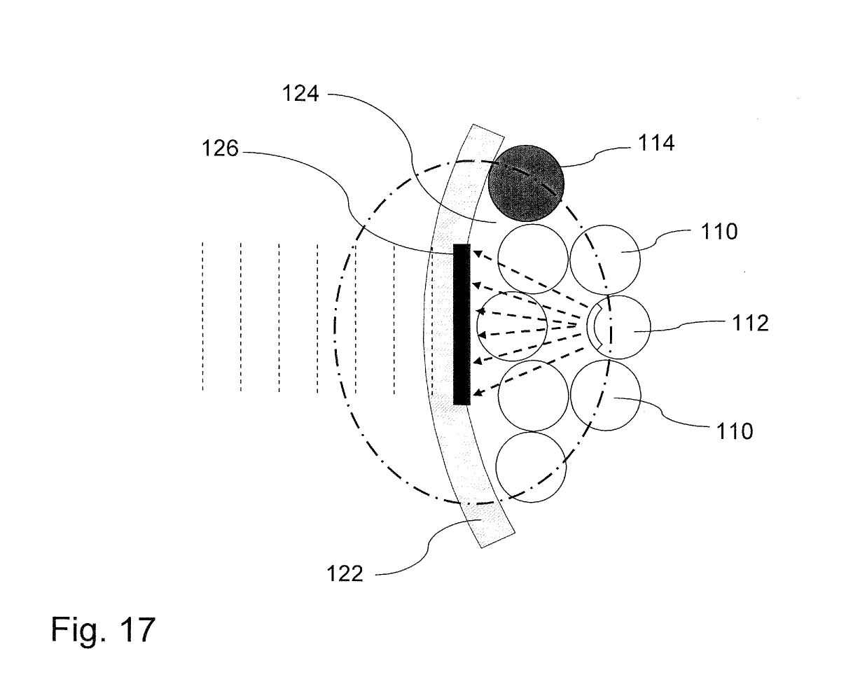

FIG. 16 illustrates another example embodiment of the distal tip of the catheter 100. The perspective view shown here is the cross-section B in FIG. 10. The embodiment here is almost the same as the one shown in FIG. 12, with the main difference being that the photoacoustic fibers 112 are positioned near the inner wall 120, as opposed to the outer wall 122, of the catheter. This arrangement provides greater distance between a photoacoustic fiber 112 and a photoacoustic layer 126, thereby allowing the laser light exiting the fiber to expand more, resulting in a larger laser spot size at the photoacoustic layer. In general, the ultrasound excitation spot on the photoacoustic layer should be much larger than the ultrasound wavelength. This reduces the amount of diffraction or angular spreading experienced by the ultrasonic wave propagating in the surrounding medium. FIG. 17 further illustrates the area marked as detail C in FIG. 16. Again the space between fibers are filled with refractive-index matching material 124 so that light exiting the side of photoacoustic fiber 112 can propagate transversely through the ablative fibers 110 without significant reflection or refraction before arriving at the photoacoustic layer.

FIGS. 13, 14 and 17 illustrated several example embodiments of light coupling from an optical fiber into a photoacoustic layer. Those skilled in the pertinent art are aware of other ways to couple light into photoacoustic layers. One example is by having a photoacoustic layer deposited on the outside wall of a thin glass collar or plate, and having a photoacoustic fiber butt-coupled to a thin edge of the glass collar or plate. The laser energy from a photoacoustic fiber would undergo multiple reflections between the two walls of the thin glass collar or plate and get absorbed by the photoacoustic layer.

The invention also encompasses a novel guidewire having imaging and ablation capabilities. Guidewires and catheters are both elongated and are typically far greater in length than they are in diameter. A guidewire differs from a catheter in that while a catheter is tubular and has a hollow core, a guidewire typically has a solid cross-section and lacks a hollow core. In every other respect, however, the teachings above and various embodiments disclosed and described may be applied to construct a novel guidewire that falls within the scope of the invention. The claims herein to a “device” therefore include both a catheter and a guidewire.

Although the invention has been described in detail, those skilled in the pertinent art should understand that they can make various changes, substitutions and alterations herein without departing from the spirit and scope of the invention in its broadest form.

What is claimed is:

1. A device for ultrasonic imaging and ablation, comprising:
a fiber-optic bundle configured to carry laser light for ultrasonic imaging, each fiber of said fiber-optic bundle having a reflective layer oriented at an acute angle with respect thereto at a distal end thereof;
an elongated member associated with said fiber-optic bundle and configured to carry energy for ablation, said energy for ablation projecting past said distal end; and
a photoacoustic layer associated with said each fiber of said fiber-optic bundle, radially separated from said elongated member and configured to receive at least some of said laser light for said ultrasonic imaging and generate ultrasonic pressure waves in response thereto.

2. The device as recited in claim 1 wherein said elongated member is at least one optical fiber in said fiber-optic bundle and said energy for ablation is laser light for ablation.

3. The device as recited in claim 1 wherein said energy for ablation is a selected one of:
radio-frequency energy,
ultrasonic energy, and
mechanical energy.

4. The device as recited in claim 1 wherein said reflective layer is located on an end face of said each fiber of said fiber-optic bundle.

5. The device as recited in claim 4 wherein said end face angles forward toward a centerline of said device and said distal cap has a frustoconical profile at a distal end thereof.

6. The device as recited in claim 1 wherein said each fiber of said fiber-optic bundle is located radially inwardly of said elongated member.

7. The device as recited in claim 1 wherein said device has a bore and further comprises a guidewire located in said bore.

8. A method of ultrasonic imaging and ablation, comprising:
causing laser light for ultrasonic imaging to be carried through a fiber-optic bundle of a device, each fiber of said fiber-optic bundle having a reflective layer oriented at an acute angle with respect thereto at a distal end thereof;
causing energy for ablation to be carried through an elongated member associated with said fiber-optic bundle, said energy for ablation projecting past said distal end; and
causing a photoacoustic layer associated with said each fiber of said fiber-optic bundle and radially separated from said elongated member to receive at least some of said laser light for said ultrasonic imaging and generate ultrasonic pressure waves in response thereto.

9. The method as recited in claim 8 wherein said elongated member is at least one optical fiber in said fiber-optic bundle and said energy for ablation is laser light for ablation.

10. The method as recited in claim 8 wherein said energy for ablation is a selected one of:
radio-frequency energy,
ultrasonic energy, and
mechanical energy.

11. The method as recited in claim 8 wherein said reflective layer is located on an end face of said each fiber of said fiber-optic bundle.

12. The method as recited in claim 11 wherein said end face angles forward toward a centerline of said device and said distal cap has a frustoconical profile at a distal end thereof.

13. The method as recited in claim 8 wherein said each fiber of said fiber-optic bundle is located radially inwardly of said elongated member.

14. The method as recited in claim 8 wherein said device has a bore and further comprises a guidewire located in said bore.

15. A device for ultrasonic imaging and ablation, comprising:
a fiber-optic bundle configured to carry laser light for ultrasonic imaging;
an elongated member associated with said fiber-optic bundle and configured to carry energy for ablation;
a distal cap having a glass element aligned with said fiber-optic bundle to receive said laser light for said ultrasonic imaging;
a reflective layer oriented at an acute angle with respect to said glass element and configured substantially to reflect said laser light for said ultrasonic imaging, said energy for ablation projecting past said reflective layer; and
a photoacoustic layer associated with said glass element and configured to receive at least some of said laser light for said ultrasonic imaging and generate ultrasonic pressure waves in response thereto.

16. The device as recited in claim 15 wherein said elongated member is at least one optical fiber in said fiber-optic bundle, said energy for ablation is laser light for ablation and said reflective layer is a dichroic layer configured substantially to transmit said laser light for ablation.

17. The device as recited in claim 15 wherein said energy for ablation is a selected one of:
radio-frequency energy,
ultrasonic energy, and
mechanical energy.

18. The device as recited in claim 15 wherein said reflective layer is located on an end face of said glass element.

19. The device as recited in claim 18 wherein said end face angles forward toward a centerline of said device and said distal cap has a frustoconical profile at a distal end thereof.

20. The device as recited in claim 15 wherein said glass element has a reflective coating located proximate said end face.

21. The device as recited in claim 15 wherein said device has a bore and further comprises a guidewire located in said bore.

22. A method of ultrasonic imaging and ablation, comprising:
causing laser light for ultrasonic imaging to be carried through a fiber-optic bundle of a device;
causing energy for ablation to be carried through an elongated member associated with said fiber-optic bundle;
causing said laser light for said ultrasonic imaging to be received into a distal cap having a glass element aligned with said fiber-optic bundle;
causing said laser light for said ultrasonic imaging to be substantially reflected off a reflective layer oriented at an acute angle with respect to said glass element;
causing said energy for ablation to be projected past said reflective layer; and
causing at least some of said laser light for said ultrasonic imaging to be received by a photoacoustic layer associated with said glass element and converted into ultrasonic pressure waves.

23. The method as recited in claim 22 wherein said elongated member is at least one optical fiber in said fiber-optic bundle, said energy for ablation is laser light for ablation and said reflective layer is a dichroic layer configured substantially to transmit said laser light for ablation.

24. The method as recited in claim 22 wherein said energy for ablation is a selected one of:
radio-frequency energy,
ultrasonic energy, and
mechanical energy.

25. The method as recited in claim 22 wherein said reflective layer is located on an end face of said glass element.

26. The method as recited in claim 25 wherein said end face angles forward toward a centerline of said device and said distal cap has a frustoconical profile at a distal end thereof.

27. The method as recited in claim 22 wherein said glass element has a reflective coating located proximate said end face.

28. The method as recited in claim 22 wherein said device has a bore and further comprises a guidewire located in said bore.
